(19) United States
(12) Patent Application Publication
(10) Pub. No.: US 2006/0223194 A1
(43) Pub. Date: Oct. 5, 2006

(54) Methods of screening for post-translationally modified proteins

(76) Inventors: Viorica Lopez-Avila, Loveland, CO (US); Carol T. Schembri, Loveland, CO (US)

Correspondence Address: AGILENT TECHNOLOGIES INC.;INTELLECTUAL PROPERTY ADMINISTRATION, LEGAL DEPT,, M/S DU404, P.O. BOX 7599, LOVELAND, CO, 80537-0599, US

(21) Appl. No.: 11/097,035

(22) Filed: Apr. 1, 2005

(51) Int. Cl. G01N 33/558

(52) U.S. Cl. 436/514

(57) ABSTRACT

The invention provides methods of analyzing a sample. In general, the methods involve: a) depositing sub-fractions of a multi-dimensionally fractionated sample into wells of a multi-well substrate; b) contacting each of the deposited sub-fractions with an array to produce a set of sub-fraction-contacted arrays; and c) interrogating the sub-fraction-contacted arrays to identify a sub-fraction containing a post-translationally modified analyte. Also provided are systems and kits for performing the subject methods.

### BACKGROUND OF THE INVENTION

Post-translational modification of a protein in a cell involves the enzymatic addition of a chemical group, e.g., a phosphate or glycosyl group, to an amino acid of that protein. Such modifications are thought to be required for maintaining and regulating protein structure and function, and abnormal post-translational events have been detected in a wide variety of diseases and conditions, including heart disease, cancer, neurodegenerative and inflammatory diseases and diabetes.

Protein phosphorylation is a type of post-translational modification used to selectively transmit regulatory signals from receptors positioned at the surface of a cell to the nucleus of the cell. The molecules mediating these reactions are predominantly protein kinases that catalyze the addition of phosphate groups onto selected proteins, and protein phosphatases that catalyze the removal of those phosphate groups. Complex biological processes such as cell cycle, cell growth, cell differentiation, and metabolism are orchestrated and tightly controlled by reversible phosphorylation events that modulate protein activity, stability, interactions and localization. Accordingly, protein phosphorylation is thought to play a regulatory role in almost all aspects of cell biology. Perturbations in protein phosphorylation, e.g., by mutations that generate constitutively active or inactive protein kinases and phosphatases, play a prominent role in oncogenesis. Serine, threonine, tyrosine, histidine, arginine, lysine, cysteine, glutamic acid or aspartic acid residues may be phosphorylated. The hydroxyl groups of serine, threonine or tyrosine residues are most commonly phosphorylated.

Protein glycosylation, on the other hand, is acknowledged as being a post-translational modification that has a major effect on protein folding, conformation distribution, stability and activity. Carbohydrates in the form of asparagine-linked (N-linked) or serine/threonine (O-linked) oligosaccharides are major structural components of many cell surface and secreted proteins. All N-linked carbohydrates are linked through N-acetylglucosamine, and most O-linked carbohydrates are attached through N-acetylgalactosamine. O-linked N-acetylglucosamine. (O-GlcNAc) is a recently identified type of glycosylation. Unlike classical O- or N-linked protein glycosylation, O-GlcNAc glycosylation involves linking a single GlcNAc moiety to the hydroxyl group of a serine or threonine residue. Increasing evidence suggests that O-GlcNAc modification is a regulatory modification similar to phosphorylation, since it is highly dynamic and rapidly cycles in response to cellular signals.

Because of the central role of post-translational modification in cell biology, much effort has been focused on the development of methods for identifying post-translationally modified proteins. A variety of methods for identifying and characterizing post-translationally modified proteins have been developed.

For example, traditional methods for analyzing phosphorylation sites involve incorporation of radioactive phosphorus into cellular phosphorylated proteins by feeding cells with ³²P ATP. The radioactive proteins can be detected during subsequent fractionation procedures (e.g., two-dimensional gel electrophoresis or high-performance liquid chromatography). Proteins thus identified can be subjected to complete hydrolysis and the phosphoamino acid content determined. The site(s) of phosphorylation can be determined by proteolytic digestion of the radiolabeled protein, separation and detection of phosphorylated peptides (e.g., by two-dimensional peptide mapping), followed by peptide sequencing by Edman degradation. These techniques are generally tedious, require significant quantities of the phosphorylated protein and involve the use of considerable amounts of radioactivity.

In recent years, affinity chromatography has become widely employed in many of methods for identifying post-translational modifications. The most widely used method involves selectively enriching phosphoproteins from a sample using immobilized metal affinity chromatography (IMAC). In this technique, metal ions, usually Fe³⁺ or Ga³⁺, are bound to a chelating support. Phosphoproteins are selectively bound to the column by the affinity of the phosphate moiety of the phosphoproteins to the metal ions of the column. The phosphoproteins can be released using high pH buffer, and subjected to mass spectrometry (MS) analysis. While this method is widely employed, it is limited because many phosphoproteins are unable to bind to IMAC columns, and bound phosphoproteins are often difficult to elute from such columns. Furthermore, these methods produce significant background signals from unphosphorylated proteins that are typically acidic in nature and therefore have affinity for the immobilized metal ions of such columns.

Accordingly, there is an ongoing need for straightforward and reliable methods to identify post-translationally modified proteins in a sample. This invention meets this need, and others.

Publications of interest include: Martin et al, (Proteomics, 2003 3:1244-55); Steinberg et al, (Proteomics, 2003 3:1128-44) and Martin et al, (Comb. Chem. High Throughput Screen., 2003 6:331-9); published US patent applications US20040180380, 20040009530, 20040119013, 20040185448, 20040086869 and 20050014197; and U.S. Pat. Nos. 6,720,157 and 5,874,219.

### SUMMARY OF THE INVENTION

The invention provides methods of analyzing a sample. In general, the methods involve: a) depositing sub-fractions of a multi-dimensionally fractionated sample into wells of a multi-well substrate; b) contacting each of the deposited sub-fractions with an array to produce a set of sub-fraction-contacted arrays; and c) interrogating the sub-fraction-contacted arrays to identify a sub-fraction containing a post-translationally modified analyte. The arrays may be optically or spatially addressable. In one embodiment, the arrays are present on a multi-array substrate. The identified sub-fraction may be subjected to mass analysis in order to determine the identity of the post-translationally modified analyte. Also provided are systems and kits for performing the subject methods. The invention finds use in a variety of different medical, research and proteomics applications.

### BRIEF DESCRIPTION OF THE FIGURES

FIG. 1 illustrates many general features of a pillar array that may be employed in the subject methods.

FIG. 2 is a flow diagram describing a representative embodiment of the subject methods.

### Definitions

Unless defined otherwise, all technical and scientific terms used herein have the same meaning as commonly understood by one of ordinary skill in the art to which this invention belongs. Still, certain elements are defined below for the sake of clarity and ease of reference.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

Components in a sample are termed “analytes” herein. In certain embodiments, the sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte.

The term “analyte” is used herein to refer to a known or unknown component of a sample, which will specifically bind to a capture agent on a substrate surface if the analyte and the capture agent are members of a specific binding pair. In general, analytes are biopolymers, i.e., an oligomer or polymer such as an oligonucleotide, a peptide, a polypeptide, an antibody, or the like. In this case, an “analyte” is referenced as a moiety in a mobile phase (e.g., fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other).

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10-100 nucleotides in length and polynucleotides of greater than 100 nucleotides in length. The term “polypeptide” refers to a polymer of amino acids of any length, including peptides that range from 6-50 amino acids in length and polypeptides that are greater than about 50 amino acids in length.

In most embodiments, the terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components that, while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of single stranded or double stranded DNA may employed. Capture agents usually “specifically bind” one or more analytes. For example, antibodies and peptides are types of capture agents.

Accordingly, the term “capture agent” refers to a molecule or a multi-molecular complex which can specifically bind an analyte, e.g., specifically bind an analyte for the capture agent, with a dissociation constant (K_{D}) of less than about 10⁻⁶ M without binding to other targets.

The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a K_{D} (dissociation constant) of less than 10⁻⁶ M, less than 10⁻⁷ M, less than 10⁻⁸ M, less than 10⁻⁹ M, usually less than about 10⁻¹⁰ M.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets. Specific binding conditions for representative capture agent/analyte interactions are well known in the art and generally involve incubating the capture agent/analyte mixture in a binding buffer, e.g., phosphate buffered saline (PBS; 137 mM NaCl, 10 mM phosphate, 2.7 mM KCl, pH 7.4) or Tris buffered saline (10 mM Tris 50 mM NaCl, pH. 7.0) for a period of time, usually from 1 to 12 hours at room temperature or 37° C., for example

As used herein, “binding partners” and equivalents refer to pairs of molecules that can be found in a capture agent/analyte complex, i.e., exhibit specific binding with each other.

The phrase “surface-bound capture agent” refers to a capture agent that is immobilized on a surface of a solid substrate, where the substrate can have a variety of configurations, e.g., a sheet, bead, or other structure, such as a plate with wells. In certain embodiments, the collections of capture agents employed herein are present on a surface of the same support, e.g., in the form of an array.

The term “pre-determined” refers to an element whose identity is known prior to its use. For example, a “predetermined analyte” is an analyte whose identity is known prior to any binding to a capture agent. An element may be known by name, sequence, molecular weight, its function, or any other attribute or identifier. In some embodiments, the term “analyte of interest”, i.e., an known analyte that is of interest, is used synonymously with the term “pre-determined analyte”.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

The term “mixture”, as used herein, refers to a combination of elements, e.g., capture agents or analytes, that are interspersed and not in any particular order. A mixture is homogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not spatially addressable. To be specific, a spatially addressable array of capture agents, as is commonly known in the art and described in greater detail below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “array” encompasses the term “microarray” and refers to an array of capture agents for binding to aqueous analytes and the like.

An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain. In some embodiments, the capture agents are not bound to the array, but are present in a solution that is deposited into or on features of the array.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations. The term “array” encompasses the term “microarray” and refers to any one-dimensional, two-dimensional or substantially two-dimensional (as well as a three-dimensional) arrangement of spatially addressable regions, usually bearing biopolymeric capture agents, e.g., polypeptides, nucleic acids, and the like.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained capture agent.

An array may be spatially addressable or optically addressable. An array is “spatially addressable” when it has multiple regions of different moieties (e.g., different capture agents) such that a region (i.e., a “feature” or “spot” of the array) at a particular predetermined location (i.e., an “address”) on the array will detect a particular sequence. Array features are typically, but need not be, separated by intervening spaces. An “optically addressable” array contains an aqueous population of capture agents that are labeled with a optically distinguishable tags. The individual species of capture agent of an optically addressable array are usually bound to the same solid substrate (e.g., a bead or plurality thereof) and are linked to an optically detectable tag (e.g., a fluorophore) so that they can be separated and distinguished from other capture agents. Optically addressable arrays of capture agents readily adaptable to the instant methods are described in greater detail in U.S. Pat. Nos. 6,649,414 and 6,524,793.

An “array layout” refers to one or more characteristics of the features, such as feature positioning on the substrate, one or more feature dimensions, and an indication of a moiety at a given location.

The term “fractionate” refers to the separation of a liquid composition into distinct, different liquid fractions via chromatography. The “fractions” of a fractionated sample each generally contain a different set of analytes, although certain analytes may be present in more than one fraction of the fractionated sample.

The term “multi-dimensionally fractionated sample” refers to a sample that has been fractioned by at least two different chromatography methods. In one exemplary embodiment provided to illustrate what is meant by this term, a “multi-dimensionally fractionated sample” is a sample that has been fractionated by ion exchange chromatography (i.e., fractionated in a first dimension) and by reverse phase chromatography (i.e., fractionated in a second dimension). In this example, the fractions produced by ion exchange chromatography are fractionated by reverse phase chromatography to produce sub-fractions. Methodologies for making multi-dimensionally fractionated samples are well known in the art (see, e.g., Apffel, A. “Multidimensional Chromatography of Intact Proteins” in Purifying Proteins for Proteomics: A Laboratory Manual, Richard Simpson (ed.), Cold Spring Harbor Press, 2003).

The term “sub-fraction” refers to a type of fraction obtained after a sample has been multi-dimensionally fractionated (i.e., fractionated by at least two different chromatography devices). A “sub-fraction” is therefore a fraction obtained by fractionation of a fraction, using a second chromatography device.

A “portion” of a liquid composition is part of a liquid composition. A portion of a liquid composition may be removed from the liquid composition (e.g., by pipetting from the composition), or portions of a liquid composition may be made by dividing the liquid composition. All of the portions of a composition generally contain the same molecules at the same relative concentrations (excluding any molecules that may have evaporated or may have been changed or removed during processing of the composition).

A “multi-array substrate” is any substrate containing a plurality of distinct arrays. The arrays of a multi-array substrate employed in the subject methods may be generally arranged in a pattern that corresponds to the wells of a multi-well plate. A multi-array substrate and a multi-well substrate may be operably engageable in that they fit together to allow contact between the individual arrays of a multi-array substrate and the corresponding individual wells of the multi-well plate. In certain embodiments, operable engagement of a multi-array substrate and a multi-well substrate provides a plurality of sealed reaction chambers.

A “pillar array”, as will be described in greater detail below, is multi-array substrate containing a plurality of spatially addressable arrays that are situated at the tops of distinct elongated elements (i.e., pillars). A pillar array is usually, although not always, operably engageable with a multi-well plate in that it is dimensioned so that the arrays at the tops of the pillars of the pillar array enter the wells of a multi-well plate when the pillar array and multi-well plate are brought together.

The term “well” encompasses any fluid-retaining structure. A well may be shallow (i.e., having fluid-retaining walls of e.g., about 0.5 mm to about 2 mm in height), or deep (i.e., greater than about 2 mm in height, e.g., greater than about 5 mm in height). Standard format 24 (4×6), 48 (6×8), 96 (8×12), 384 (16×24) and 1536 (32×48) multi-well plates having well walls of any height, and the multi-well MALDI sample plates described in US20040119013 and US20040185448, are representative multi-well plates that may be employed in the subject methods.

A “capture agent that binds a post-translationally modified analyte” is any capture agent (e.g., a polypeptide such as an antibody) that can detectably bind a post-translationally modified analyte (e.g., a post-translationally modified polypeptide). Capture agents that specifically bind post-translationally modified analytes generally do not detectably bind non-post-translationally modified analytes.

If a first element is “bound to” a second element, the binding between those elements may be either direct or indirect (e.g., by means of third element that simultaneously binds to both the first and the second elements). The linkage between a first element bound to a second element may be covalent or non-covalent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### DETAILED DESCRIPTION OF THE INVENTION

The invention provides methods of analyzing a sample. In general, the methods involve: a) depositing sub-fractions of a multi-dimensionally fractionated sample into wells of a multi-well substrate; b) contacting each of the deposited sub-fractions with an array to produce a set of sub-fraction-contacted arrays; and c) interrogating the sub-fraction-contacted arrays to identify a sub-fraction containing a post-translationally modified analyte. The arrays may be optically or spatially addressable. In one embodiment, the arrays are present on a multi-array substrate. The identified sub-fraction may be subjected to mass analysis in order to determine the identity of the post-translationally modified analyte. Also provided are systems and kits for performing the subject methods. The invention finds use in a variety of different medical, research and proteomics applications.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, the subject methods are described first, followed by a description of a system for analyzing a sample in which the subject methods may be employed. Kits for use in performing the subject methods will then be described.

Methods of Sample Analysis

The instant methods of sample analysis may employ optically addressable arrays or spatially addressable arrays and in certain embodiments the instant methods of sample analysis employ a multi-array substrate. In representative embodiments, a multi-array substrate is employed. Multi-array substrates that may be employed in the subject methods include pillar arrays (as will be described in great detail below), so called “chip plates” that contain a plurality of test wells each having an array (described in great detail in U.S. Pat. No. 5,874,219), and other types of multi-array substrates that can be operably engaged with a multi-well substrate. Exemplary methods of the instant invention that employ pillar arrays are described in great detail below. The exemplary methods described are readily adapted for use with other multi-array substrates, including chip plates, and should not be construed as limiting the claimed invention to pillar arrays.

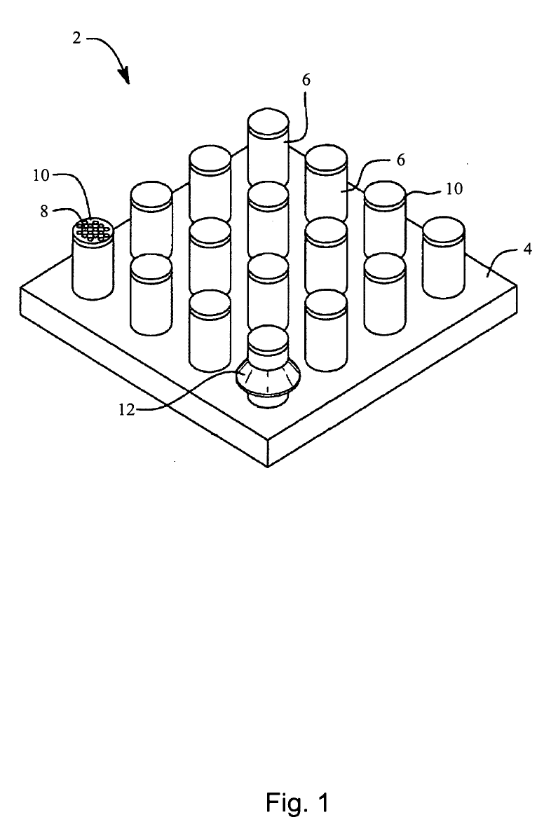

A pillar array, an exemplary multi-array substrate that may be employed in the subject methods, is described in great detail in U.S. patent application Ser. No. 10/285,756 (published as US20040086869 and incorporated in its entirety for all purposes). FIG. 1 shows several general features of a pillar array that may be employed in the subject methods. With reference to FIG. 1, a pillar array 2, as employed in the instant methods, is a multi-array device containing a foundation support 4 and a plurality of pillars (or “prongs” as they are sometimes called) extending from the foundation support 6. The pillars of a pillar array each contain a spatially addressable capture agent array 8 attached at their distal end (where the proximal end of a pillar is affixed to the foundation substrate). There are many ways to make a pillar array. For example, as described in 10/285,756 and as will be described in greater detail below, pillar arrays may be made by first fabricating a plurality of arrays on a flexible substrate, and then attaching array-containing portions of the flexible substrate to the distal ends of the subject pillars. Accordingly, in certain embodiments, the subject arrays may be affixed to the pillars of a pillar array via a flexible substrate 10. Alternatively, the arrays may be built or deposited directly on the distal of each pillar.

In an alternative embodiment, the multi-array substrates that are planar (as described in U.S. Pat. No. 6,682,702 and U.S. patent application Ser. No. 10/766,766 (published as US20040208800)) may be employed.

In many embodiments of the instant methods, a multi-array substrate is operably engageable with a multi-well plate. The pillars of a pillar array may therefore be arranged in a pattern corresponding to that of the wells of a multi-well plate and the arrays of a pillar array enter into the respective wells of a multi-well plate when the pillar array and multi-well plate are engaged. Likewise, the wells of a chip plate may be arranged in a pattern corresponding to that of the wells of a multi-well plate, and the respective chip plate wells and multi-well plate well may seal with each other when the plates are engaged. The arrays of a chip array may be combined with the contents of the respective wells of a multi-well plate when the chip plate and multi-well plate are inverted.

A multi-array substrate may be configured to engage with any multi-well plate, including multi-well plates in a 4×6, 8×12, 16×24, 32×48 format, as well as any of the multi-well MALDI sample plate described in US20040119013 or US20040185448. In particular embodiments, the pillars of a pillar array may contain a seal feature (e.g., a shoulder feature 12) that makes contact with a seal element (e.g., a gasket) that surrounds the opening of a well when the pillar array and multi-well plate are engaged. Likewise, the wells of a chip plate may each be surrounded by a seal feature that engages with a seal element (e.g., a gasket) that surrounds the opening of each of the wells of the multi-well plate well when the chip plate and multi-well plate are engaged

In general terms, the subject method involves: a) fractionating a sample in at least two dimensions (i.e., using at least two different chromatography methods) to produce a set of sub-fractions that are deposited into the wells of a multi-well plate, b) operably engaging the multi-well plate with a multi-array substrate, e.g., a pillar array, containing arrays of post-translationally modified analyte-capture agents; and c) evaluating the multi-array substrate to identify a sub-fraction containing post-translationally modified analytes. The mass of analytes in an identified sub-fraction may be assessed. In one embodiment, an identified sub-fraction is ionized and subjected to mass spectrometry in order to analyze the masses of analytes in that sub-fraction.

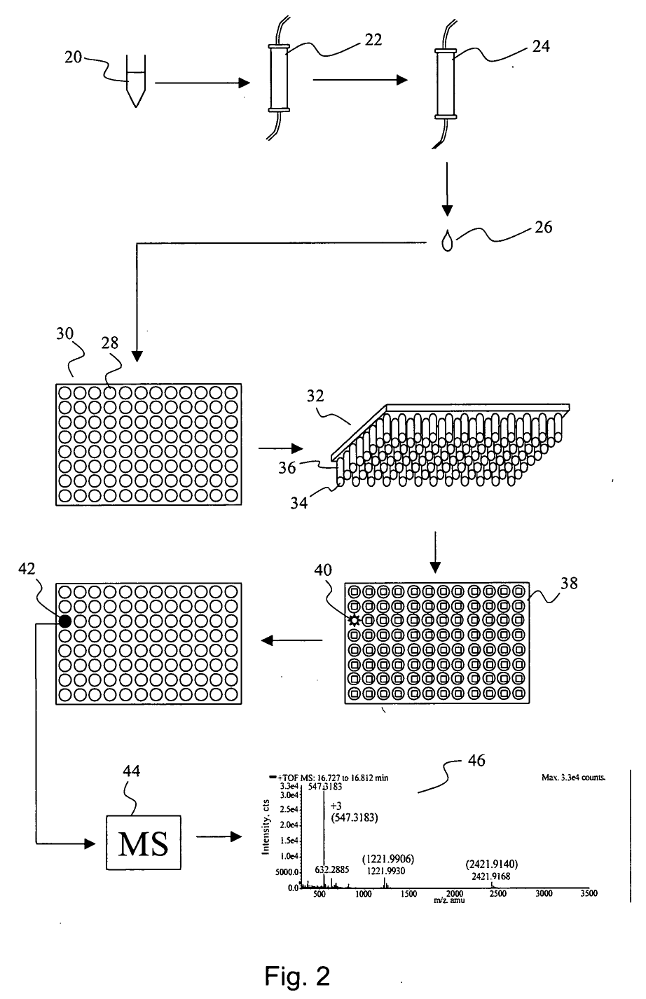

With reference to FIG. 2, showing an exemplary embodiment not intended to limit the invention, the method may involve producing a multi-dimensionally fractionated sample by fractionating a sample 20 using a first chromatography device 22 to produce a plurality of fractions, and fractionating those fractions using a second chromatography device 24 to produce a set of sub-fractions 26. The sub-fractions are individually placed into the wells 28 of a multi-well plate 30, either directly or indirectly via an addressable storage system. The placement of sub-fractions 26 into the wells 28 of multi-well plate 30 may, in certain embodiments, be done using a fraction collector operably connected to the second chromatography device (not shown in FIG. 2).

Exemplary multi-array substrate, pillar array 32, containing arrays of post-translationally modified analyte-capture agents 34 that are upon pillars 36, is operatively engaged with the multi-well plate such that the arrays of pillar array 32 are in contact with (e.g., submersed in) the sub-fractions present in the wells. The pillar array and the multi-well plate are maintained under conditions suitable for binding of post-translationally modified analytes in the deposited sub-fractions to the arrays of post-translationally modified analyte-capture agents. After the pillar array has been contacted with the multi-well plate washed as necessary, and, if needed, exposed to secondary antibodies or other labeling techniques, pillar array 38 is interrogated (i.e., read or scanned) to identify a sub-fraction containing a post-translationally modified analyte 40. After a sub-fraction containing a post-translationally modified analyte is identified, the well of the multi-well plate containing that sub-fraction 42 is identified, and a portion of that sub-fraction is subjected to mass analysis, e.g., using mass spectrometry 44 to produce data 46 regarding the identity of the post-translationally modified analyte in that sub-fraction, e.g., a post-translationally modified polypeptide. The identity of the analyte bound by the binding agent can be determined using this data.

In an alternative embodiment, the arrays employed in the instant methods may be optically addressable arrays (or so called “bead arrays”) and may contain capture agents linked to an optically detectable tag (e.g., a bead). Exemplary optically addressable array systems include xMAP technology by Luminex Corporation (Austin Tex.), Qbead microspheres by Quantum Dot Corporation (Hayward, Calif.) and the like.

In embodiments of the instant methods that employ optically-addressable arrays, one to several thousand or more optically-tagged beads or spheres each comprising at least one capture agent is added to each well of the microplate and allowed to react under conditions suitable for binding of the beads to analytes in the sub-fraction. After a suitable amount of time, the beads are separated from the samples (e.g., using paramagnetism, centrifugation, aspiration or another separation approaches specified by the bead manufacturers), and washed. The beads may then be contacted with a secondary antibody or a label and read to determine which capture agent is bound to a post-translationally modified analyte.

In describing these methods in greater detail, the multi-dimensional fractionation methods will be described first, followed by a discussion of the arrays. Finally, the subject methods of using a arrays to identify sub-fractions containing post-translationally modified polypeptides will be described.

Multi-Dimensional Fractionation

The subject methods of sample analysis involve multi-dimensional fractionation of a sample. In general, multi-dimensional fractionation methods employ at least two different liquid chromatography devices (termed herein as a “first” chromatography device and “second” chromatography device), and the sample is fractionated using both of those devices. A sample is fractionated by a first chromatography device to produce fractions, and those fractions are themselves fractionated by a second chromatography device to produce sub-fractions. The sub-fractions produced by the second chromatography device are then used in the remainder of the methods, as will be discussed in greater detail below.

For many purposes, any two or more different liquid chromatography devices may be used to multi-dimensionally fractionate a sample. Accordingly, there are many liquid chromatography devices that may be employed in the subject methods including, but not limited to: a) hydrophobic interaction chromatography devices (e.g., normal or reverse phase chromatography devices that employ a hydrophobic column, for example a C4, C8 or C18 column), b) ion exchange chromatography devices (e.g., anion exchange or cation exchange (including strong cation exchange) devices that employ, for example, a diethyl aminoethyl (DEAE) or carboxymethyl (CM) column), c) affinity chromatography devices (e.g., any chromatography device having a column linked to a specific binding agent such as a polypeptide, a nucleic acid, a polysaccharide or any other molecule such as, for example a chelated metal (e.g., chelated Fe³⁺ or Ga³⁺) and IMAC columms), and d) gel filtration chromatography devices (e.g., any chromatography device containing a size excluding gel such as SEPHADEX™ or SEPHAROSE™ of any pore size) that separate analytes in a sample on the basis of their size. High performance liquid chromatography (HPLC) or capillary chromatography devices are employed in certain embodiments of the invention.

The particular chromatography conditions employed with any of the above types of chromatography devices (e.g., the binding, wash or elution buffers used, the salt or solvent gradients used, whether or step or continuous gradient is used, the exact column used, and the run-time etc.), are well known in the literature and are readily adapted to the instant methods without undue effort.

The first and second chromatography devices employed in the subject methods are generally “different” to each other in that they use different physical properties to separate the analytes of a sample. Analyte size, analyte affinity to a substrate, analyte hydrophobicity and analyte charge are exemplary properties that are different to each other. Accordingly, a sample may be first fractionated using a device selected from a hydrophobic interaction chromatography device, an ion exchange chromatography device, an affinity chromatography device or a gel filtration chromatography device to produce fractions, and the resultant fractions are then themselves fractionated by a different device. In one exemplary embodiment, a sample is first subjected to ion exchange chromatography to produce fractions, and those fractions are subjected to reverse phase chromatography to produce sub-fractions.

The number of fractions produced by each of the chromatography devices employed may vary depending on the complexity of the sample to be analyzed and the particular fractionation devices used. In certain embodiments, the first chromatography device produces at least 5 (e.g., at least 10, at least 50, at least 100, at least 200, at least 500, usually up to about 500 or 1,000 or more) fractions, and each of those fractions is further fractionated into at least 5 (e.g., at least 10, at least 50, at least 100, at least 200, at least 500, usually up to about 500 or 1,000 or more) sub-fractions by the second chromatography device. In general, a sample may be multi-dimensionally fractionated into any number of sub-fractions (e.g., at least 50, at least 100, at least 500, at least 1,000, at least 5,000 or at least 10,000 usually up to about 50,000 or 100,000 fractions or more). In certain embodiments, the sub-fractions of a sample may contain, on average, less than about 10 (e.g., about 1, 2, 4, 6 or 8) different polypeptides.

In general, multi-dimensional fractionation systems readily adaptable for employment in the instant methods are known in the art. Further details of these multi-dimensional fractionation methods may be found in Wang et al. (Mass Spectrom Rev. 2004 Jun. 30; Epub ahead of print); Wang et al. (J. Chromatogr. 2003 787:11-8); Issaq et al. (Electrophoresis 2001 22:3629-38); Wolters et al. (Anal Chem. 2001 73:5683-90); and Link (Trends in Biotechnology 2002 20:S8-S13), for example.

As is known in the art, the output of a first chromatography device of a multi-dimensional chromatography system may be linked to the input of the second chromatography device of the system. In such a system, the fractions produced by the first device are further fractionated by the second device immediately after they are input into the second device from the first device. Accordingly, multi-dimensional fractionation of a sample may be continuous in that the devices employed are operating at the same time. In particular embodiment, the devices employed in a subject multi-dimensional fractionation system may be present within the same housing.

The sub-fractions of a sample may be individually deposited into the wells of a multi-well plate using a fraction collector. In certain embodiments, the collected sub-fractions may be concentrated, stored and/or mixed with other reagents (e.g., capture agent/analyte binding buffer such as salt, PBS or Tris-buffered saline) prior to use.

Further, the deposited sub-fractions may be directly or indirectly detectably labeled prior to use. A directly detectable label is a label that provides a directly detectable signal without interaction with one or more additional chemical agents. Examples of directly detectable labels include fluorescent labels. Indirectly detectable labels are those labels which interact with one or more additional members to provide a detectable signal. In this latter embodiment, the label may be a member of a signal producing system that includes two or more chemical agents that work together to provide the detectable signal. Examples of indirectly detectable labels include biotin, streptavidin or digoxigenin, which can be detected by a binding partner (e.g., streptavidin or an antibody or the like) coupled to a fluorochrome, for example.

Methods of labeling analyte samples for use in array-based experiments are generally well known in the art and are described in, for example, Zhu et al (Science, 2001 293: 2101-2105), Huang et al (Proc. Natl. Acad. Sci., 2004 101:16594-9), Saviranta et al (Clin. Chem., 2004 50:1907-20), Ge et al (Nucleic Acids Res., 2000 28:e3); Lin et al, (Cancer Lett. 2002 187:17-24), Anderson et al, (Brain 2003 126:2052-64) and Ivanof et al, (Mol. Cell Proteomics, 2004 3:788-95). These art-known methods are readily adapted to the instant methods.

In particular embodiments, greater than 0.1% and less than about 0.5%, less than about 1%, less than about 3%, less than about 5%, less than about 10% or less than about 20% of the analytes (e.g., polypeptides) in a particular sub-fraction are labeled.

Arrays

As mentioned above, the instant methods employ a plurality of optically or spatially addressable arrays. In certain embodiments a multi-array substrate, e.g., a pillar array is operably engaged with a multi-well plate such that the arrays of the multi-array substrate are contacted with the deposited sub-fractions. In another embodiment, optically addressable arrays are deposited into the wells of a subject multi-well plate such that each of the arrays contacts a deposited sub-fraction. The arrays employed in the instant methods generally contain capture agents that specifically bind to post-translationally modified analytes, e.g., phosphorylated or glycosylated polypeptides, but not to non-post-translationally modified analytes, e.g., non-phosphorylated or non-glycosylated polypeptides), as well as controls that may bind to particular analytes, regardless of their post-translational modification status (i.e., may bind to both the post-translationally modified and on-translationally modified forms of an analyte).

A variety of post-translationally modified analyte capture agents may be employed in the subject methods. In particular embodiments an antibody may be used. For example, to identify phosphoproteins (i.e., polypeptides to which a phosphate group has been added), any one or more of a variety of labeled anti-phosphotyrosine, anti-phosphoserine or anti-phosphothreonine antibodies may be used. Such antibodies may be purchased from a variety of different manufacturers, including Research Diagnostics Inc. (Flanders N.J.), Zymed Laboratories, Inc. (San Francisco, Calif.), PerkinElmer (Torrance, Calif.) and Sigma-Aldrich (St. Louis, Mo.). Likewise, to identify glycoproteins, one or more of a variety of anti-glycoprotein antibodies may be employed (see product literature for Novus (Littleton, Colo.) and Sigma-Aldrich (St. Louis, Mo.), for example).

A post-translationally modified analyte capture agent employed in the subject methods may be specific (i.e., may bind to a single species of a post-translationally modified analyte, e.g., a particular phosphorylated or glycosylated polypeptide) or non-specific (i.e., may bind to multiple species of a post-translationally modified analyte, e.g., a plurality of different phosphorylated or glycosylated polypeptides).

In certain embodiments of the invention, the capture agents are proteinaceous capture agents, methods for the making of which are generally well known in the art. For example, polypeptides may be produced in bacterial, insect or mammalian cells (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) using recombinant means, isolated, and deposited onto a suitable substrate.

Capture agents may be selected based on their binding to pre-determined post-translationally modified analytes in a sample. Accordingly, in certain embodiments of the subject methods, the pre-determined analytes and the capture agents that bind those analytes may be selected prior to starting the subject methods. In other embodiments, the capture agents are not pre-determined and their binding specificity may be unknown.

Capture agents may be chosen using any means possible. For example, sets of capture agents present on an array may bind to proteins of a particular signal transduction, developmental or biochemical pathway, post-translationally modified proteins having similar biological functions, post-translationally modified proteins of similar size or structure, or they may bind post-translationally modified proteins that are known markers for a biological condition or disease. Capture agents may also be chosen at random, or on the availability of capture agents, e.g., if a capture agent is available for purchase, for example. In some embodiments, a capture agent may be chosen purely because it is desirable to know whether a particular post-translationally modified polypeptide is present in a sample. The analyte for a capture agent does not have to be known for the capture agent to be present on an array employed in the subject methods.

Further, since the capture agents are chosen using any means possible, there is no requirement that any or all of the analytes for those capture agents are present in a sample to be analyzed. In fact, since the subject methods may be used to determine the presence or absence of an analyte in a sample, as well as the post-translational modification status of an analyte in a sample, only a fraction or none of the analytes may be present in a sample to be analyzed.

In particular embodiments, capture agents are monoclonal antibodies, although any molecule that can specifically bind a post-translationally modified analyte, e.g., other types of proteins, such as members of known binding partner pairs, antibodies such as phage display antibodies and antibody fragments or the like, may be used. Monoclonal antibodies that specifically bind to post-translationally modified analytes are well known in the art and may be made using conventional technologies (see, e.g., Harlow and Lane, Antibodies: A Laboratory Manual Cold Spring Harbor Laboratory, Cold Spring Harbor, N.Y. (1989)). Monoclonal antibodies that specifically bind to known post-translationally modified analytes may also be purchased from a number of antibody suppliers such as Santa Cruz Biotechnology, Santa Cruz, Calif. and Epitomics, Inc., Burlingame, Calif. Antibody fragments and phage display antibodies are also well known in the art and are readily employed in the subject methods.

Methods for making arrays of polypeptides using contact and inkjet (i.e., piezoelectric) deposition methods are generally well known in the art (see e.g., U.S. Pat. Nos. 6,372,483, 6,352,842, 6,346,416 and 6,242,266; MacBeath and Schreiber, Science (2000) 289:1760-3). Specific methods for producing polypeptide arrays are also found in Zhu et al (Science, 2001 293: 2101-2105); Huang et al (Proc. Natl. Acad. Sci., 2004 101:16594-9); Saviranta et al (Clin. Chem., 2004 50:1907-20); Ge et al (Nucleic Acids Res., 2000 28:e3); Lin et al, (Cancer Lett. 2002 187:17-24); Anderson et al, (Brain 2003 126:2052-64) and Ivanof et al, (Mol. Cell Proteomics, 2004 3:788-95). Methods for producing flexible arrays, i.e., arrays of capture agents on a flexible substrate, and pillar arrays are also well known and described in a variety of publications, including U.S. patent application Ser. Nos. 10/766,766 (filed on Jan. 27, 2004 and published as US20040208800), 10/286,089 (filed on Oct. 31, 2002 and published as US20040087033), 10/286,090 (filed on Oct. 31, 2002 and published as US20040087009), 10/285,759 (filed on Oct. 31, 2002 and published as US20040087008), 10/286,117 (filed on Oct. 31, 2002 and published as US20040086871), 10/285,756 (filed on Oct. 31, 2002 and published as US20040086869) and 10/286,319 (filed on Oct. 31, 2002 and published as US20040086424). Methods of making chip plates are described in U.S. Pat. No. 5,874,219. The methods described in the above-referenced publications are readily adapted to the methods described herein, and are incorporated by reference in their entireties for all purposes.

The subject arrays may generally comprises a plurality (i.e., at least two, e.g., at least 5, at least 10, at least 50, at least 100, at least 500 and, in certain embodiments, up to 1,000, 10,000 or 50,000 or more) of spatially or optically addressable features each containing one or more capture agents. In certain embodiments, there may be at least 50 (e.g., 100 or more) antibodies to particular post-translationally modified polypeptides, as well as a plurality of control antibodies. In certain embodiments, a single species of polypeptide may be present in each of the features of a subject array. However, depending on the precise methodology employed, a feature may contain a mixture of different polypeptides. As mentioned above, the arrays of a multi-array substrate may contain capture agents that detectably bind to analytes that are post-translationally modified and do not detectably bind to non-post-translationally modified analytes, as well as control capture agents that may detectably bind to analytes regardless of their post-translational modification status (i.e., may detectably bind to a post-translationally modified and a non-post-translationally modified version of the same analyte).

The individual arrays employed in the instant methods may be identical or different to each other.

Methods of Sample Analysis

If a multi-array substrate is employed in the instant methods, it is operably engaged with the above-described multi-well plate, and the arrays of the multi-array substrate are thereby contacted with the sub-fractions. The multi-array substrate and multi-well plate, once engaged, are maintained under conditions suitable for binding of the arrayed capture agents to any post-translationally modified analytes in the sub-factions.

As discussed above, in certain embodiments, the wells of a multiwell plate may contain a first sealing element, e.g., a gasket surrounding the entrance of the wells, that makes contact with a corresponding second sealing element, e.g., a shoulder element, that is present on wells or pillars the multi-array substrate. Operable engagement of such a multi-array substrate and multi-well plate contacts the first sealing element with the second sealing element, sealing the opening of the wells of the multi-well plate to produce a plurality of sealed reaction chambers that are gas and/or liquid tight. Accordingly, in certain embodiments, the instant methods include contacting the arrays of the multi-well plate with the sub-fractions in sealed reaction chambers. As mentioned above, the multi-well plate and the multi-array substrate, once engaged, may be inverted or agitated to facilitate contact between the sub-fractions and the arrays.

Upon contacting a sub-fraction with an array of capture agents under conditions suitable for specific binding of the analytes in the sub-fractions to the capture agents, capture agent/analyte complexes are formed if post-translationally modified analytes corresponding to the capture agents are present in the sub-fraction. As discussed above, it is not required that any complexes form since the post-translationally modified analytes may not be present in the sub-fraction tested.

After the arrays of the multi-array substrate have been contacted with the sub-fractions for a suitable amount of time, unbound analytes may be separated from the array by a separation step, e.g., a washing step, where any analytes that are not specifically bound to capture agents are washed away and usually discarded. Washing may be done in capture agent/analyte binding buffer, as described above. In certain embodiments, washing may be performed by disengaging the multi-array substrate from the multi-well plate containing sub-fractions, and operably engaging the multi-well substrate with a second multi-well plate containing wash buffer, for example.

Depending on how the sub-fractions are labeled (i.e., whether they are directly or indirectly labeled) the multi-array substrate may then be read (if the sub-fractions are directly labeled) or contacted with a second member of a signal producing system (if the sub-fractions are indirectly labeled) prior to being read. Again, the arrays of a multi-array substrate may be contacted with a second member of a signal producing system by operably engaging the multi-array substrate with a multi-well plate containing the second member of the signal producing system. In one embodiment of interest, the analytes of a sub-fractions are biotinylated using known methods prior to contact with the arrays, and the analytes bound to the arrays are detected by contacting the arrays with an optically detectable streptavidin molecule (e.g., a streptavidin molecule linked to a fluorescent moiety such as a cyanine dye).

A sub-fraction that contains a post-translationally modified analyte is identified by interrogating the multi-array substrate, e.g., reading the multi-array substrate using an array reader (for example, an array scanner). Details of scanners and scanning procedures that may be employed in the subject methods are found in U.S. Pat. Nos. 6,806,460, 6,791,690 and 6,770,892, for example. The pattern of signals obtained from an array of a multi-array substrate indicates whether the sub-fraction corresponding to that array (i.e., the sub-fraction to which the array was contacted) contains a post-translationally modified analyte. In general, a significant (i.e., greater than background) fluorescent signal from a feature containing a capture agent for a post-translationally modified analyte indicates that the sub-fraction with which the array containing that feature made contact contains a post-translationally modified analyte.

Once such a sub-fraction containing a post-translationally modified analyte is identified, a portion of that sub-fraction may be subjected to mass analysis, e.g., mass spectrometry analysis, to produce data. The data may be analyzed to identify the analyte of interest.

In certain embodiments, a portion (e.g., 100 nl, 500 nl, 1 μl, 2 μl, 5 μl, usually up to 10 μl or 100 μl or more) of an identified sub-fraction is removed from the multi-well plate (or a duplicate thereof), the analytes of the removed portion are ionized and the resultant ions are investigated by mass spectrometry.

In other embodiments, particularly those in which the sub-fractions are deposited directly into the wells of a MALDI sample plate containing fluid-retaining structures, the sub-fractions may be mixed with solvent and allowed to crystallize on the MALDI plate prior to ionization and subsequent analysis.

The analytes of a sub-fraction of interest are analyzed using any mass spectrometer that has the capability of measuring analyte, e.g., polypeptide, masses with high mass accuracy, precision, and resolution. Accordingly, the isolated analytes may be analyzed by any one of a number of mass spectrometry methods, including, but not limited to, matrix-assisted laser desorption ionization time-of-flight mass spectrometry (MALDI-TOF), triple quadrupole MS using either electrospray MS, electrospray tandem MS, nano-electrospray MS, or nano-electrospray tandem MS, as well as ion trap, Fourier transform mass spectrometry, or mass spectrometers comprised of components from any one of the above mentioned types (e.g., quadrupole-TOF). For example, isolated analytes may be analyzed using an ion trap or triple quadrupole mass spectrometer. In many embodiments, MALDI-TOF instrument are used because they yield high accuracy peptide mass spectrum. If MALDI methods are used, the portion to be ionized is may be concentrated on the MALDI sample plate using standard technology, e.g., repeated sample spotting followed by evaporation, to a suitable concentration, e.g., 1-10 pMole/μL. In other embodiments, a liquid sample is ionized using an electrospray system. In certain cases it may be desirable to identify a particular analyte in a sub-fraction, in which case techniques such as selective ion monitoring (SIM) may be employed.

The output from the above analysis contains data relating to the mass, i.e., the molecular weight, of analytes in the identified sub-fraction, and their relative or absolute abundances in the sample.

The analyte masses obtained from mass spectrometry analysis may be analyzed to provide the identity of the analyte. In one embodiment, the obtained masses are compared to a database of molecular mass information to identify the analyte. In general, methods of comparing data produced by mass spectrometry to databases of molecular mass information to facilitate data analysis is very well in the art (see, e.g., Yates et al, Anal Biochem. 1993 214:397-408; Mann et al, Biol Mass Spectrom. 1993 22:338-45; Jensen et al, Anal Chem. 1997 D69:4741-50; and Cottrell et al., Pept Res. 1994 7:115-24) and, as such, need not be described here in any further detail.

Accordingly, the identity of an analyte in a sub-fraction of interest may be obtained using mass spectrometry. Further details of exemplary mass spectrometry systems that may be employed in the subject methods may be found in U.S. Pat. Nos. 6,812,459, 6,723,98, 6,294,779 and RE36,892.

As is well known in the art, for each analyte, information obtained using mass spectrometry may be qualitative (e.g., showing the presence or absence of an analyte, or whether the analyte is present at a greater or lower amount than a control analyte or other standard) or quantitative (e.g., providing a numeral or fraction that may be absolute or relative to a control analyte or other standard). Accordingly, the relative levels of a particular analyte in two or more different sub-fractions may be compared.

In certain embodiments, at any stage of the methods set forth above, the analytes may be cleaved into analyte fragments prior to mass analysis. For example, the analytes of an identified sub-fraction of interest may be cleaved prior to mass analysis to provide sequence information. In certain embodiments, cleaved and uncleaved portions of a sub-fraction of interest may be separately assessed by mass analysis to determine the identity of an analyte therein. Fragmentation of analytes can be achieved by chemical means, e.g., using cyanogen bromide or the like, enzymatic means, e.g., using a protease enzyme such as trypsin, chymotrypsin, papain, gluc-C, endo lys-C, proteinase K, carboxypeptidase, calpain, subtilisin or pepsin or the like, or physical means, e.g., sonication or shearing. The cleavage agent can be immobilized in or on a support, or can be free in solution.

Likewise, at any point in the above-recited methods, a portion of an identified sub-fraction may be treated with a kinase (e.g., a specific or non-specific serine, threonine or tyrosine kinase) or a phosphatase (e.g., a specific or non-specific phospho-serine, phospho-threonine or phospho-tyrosine phosphatate such as an alkaline phosphatase) to verify that a particular phosphoprotein is present or absent in a sub-fraction. For example, an sub-fraction may be treated with a kinase or phosphatase to add or remove phosphate groups from polypeptides of the array. The presence of a particular phosphoprotein in a particular sub-fraction can be verified by comparing results obtained using treated and untreated sub-fractions. In one embodiment, prior to mass analysis, a portion of a sub-fraction identified as containing a phosphoprotein can be treated with a kinase or phosphatase to verify that the sub-fraction does, indeed, contain a phosphoprotein. In certain embodiments, a portion of a sub-fraction or an array may be first treated with a phosphatase, and then treated with a kinase to verify the presence of a phosphoprotein. Such methods are readily adapted from those methods already known in the art, such as those of Zhang et al (Anal Chem. 1998 70:2050-9).

Numerical data corresponding to the amount of a post-translationally modified analyte associated with the features of an array may be produced using feature extraction software. Amounts of signal may be measured as an quantitative (e.g., absolute) value of signal, or a qualitative (e.g., relative) value of signal, as is known in the art.

The identity of post-translationally modified analytes in a sample can be determined using the above methods.

System for Sample Analysis

In accordance with the above, the invention further provides a system for sample analysis. In general, the subject system contains: a) a multi-dimensional sample fractionation system for producing sub-fractions of a sample, b) a plurality of arrays, e.g., a multi-array substrate such as a planar or pillar array containing capture agents that specifically bind to post-translationally modified analytes, or optically addressable arrays and c) a system for assessing analyte mass. In certain embodiments, a subject multi-dimensional sample fractionation system may contain an ion exchange chromatography device and reverse phase chromatography device that may be linked to each other, and, in particular embodiments, may also contain a fraction collector for depositing sub-fractions into multi-well plates. The system for assessing binding may contain a device for depositing material on an flexible substrate to form a flexible array (i.e., an “arrayer”) and a multi-array substrate reader. The system for assessing analyte mass may be a mass spectrometer system containing an ion source, a mass spectrometer (e.g., a TOF spectrometer or an ion trap), and any necessary ion transport and detection devices present therein.

The above system and methods may be performed by hand, i.e., manually. However, in certain embodiments, the subject methods may be performed using an automated system. An exemplary automated system for analyzing a sample contains the above-recited components, as well as a robot for transferring multi-vessel storage units from one place to another, and pipetting robots. Suitable pipetting robots include the following systems: GENESIS™ or FREEDOM™ of Tecan (Switzerland), MICROLAB 4000™ of Hamilton (Reno, Nev.), QIAGEN 8000™ of Qiagen (Valencia, Calif.), the BIOMEK 2000™ of Beckman Coulter (Fullerton, Calif.) and the HYDRA™ of Robbins Scientific (Hudson, N.H.).

Utility

The subject methods may be employed in a variety of diagnostic, drug discovery, and research applications that include, but are not limited to, diagnosis or monitoring of a disease or condition (where the degree of post-translational modification of a particular analyte is a marker for the disease or condition), discovery of drug targets (where the analyte is differentially post-translationally modified in a disease or condition and may be targeted for drug therapy), drug screening (where the effects of a drug are monitored by assessing the level of post-translational modification of an analyte), determining drug susceptibility (where drug susceptibility is associated with a particular profile of post-translational modifications) and basic research (where is it desirable to identify the presence of a post-translationally modified analyte in a sample, or, in certain embodiments, the relative levels of a post-translationally modified analyte in two or more samples).

In particular embodiments, the instant methods may be used to identify post-translationally modified polypeptides, including polypeptides that have been phosphorylated or glycosylated. In these embodiments, a sample is analyzed using the above methods, and the identity of some or all of the post-translationally modified polypeptides in the sample can be determined. In certain embodiments, the subject methods may be employed to produce a “profile” of post-translationally modified polypeptides for a sample.

In certain embodiments, a sample may be analyzed to determine if a particular post-translationally modified polypeptide is present in the sample.

In other embodiments, relative post-translational modification status of an analyte of two or more different samples may be obtained using the above methods, and compared. In these embodiments, the results obtained from the above-described methods are usually normalized to the total amount of analyte present (as indicated by control capture agents), and compared. This may be done by comparing ratios, as described above, or by any other means. In particular embodiments, the post-translational modification profiles of two or more different samples may be compared to identify post-translational modification events that are associated with a particular disease or condition (e.g., a phosphorylation or glycosylation event that is induced by the disease or condition and therefore may be part of a signal transduction pathway implicated in that disease or condition).

The different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells. The subject methods are particularly employable in methods of detecting the phosphorylation status of phosphorylated serum proteins.

Accordingly, among other things, the instant methods may be used to link certain post-translational modifications (i.e., a certain modification of a certain protein) to certain physiological events.

In particular embodiments, the subject methods may be used to establish cellular signaling pathways that are employed to transmit signals in a cell (e.g., from the exterior or interior of the cell to a cell nucleus, or from one protein in a cell to another, directly or indirectly). For example, the subject methods may be employed to determine the phosphorylation status of a protein in a cell (e.g., determine how much of a particular protein is phosphorylated at any moment in time), thereby indicating the activity of the kinase or phosphatase for which that protein is a substrate, even if the identity of the kinase or phosphatase is unknown. The substrates for a particular kinase or phosphatase may be identified by virtue of the fact that they should be phosphorylated or dephosphorylated by the same stimulus, at the same point in time. A signal transduction pathway for a particular stimulus may be determined by identifying all of the phosphorylation/dephosphorylation events for a particular stimulus, and determining when those events occur. Certain post-translational modifications that occur before other post-translational modifications (e.g., immediately after a stimulus) are generally upstream in a signal transduction pathway, whereas other post-translational modifications that occur after other post-translational modifications (e.g., long after a stimulus) are generally at the end of a signal transduction pathway.

In one embodiment, the invention provides a method of screening for an agent that modulates post-translational modification. The method generally comprises contacting a candidate agent with a sample and assessing the sample according to the above-recited methods. In certain embodiments, the results from this assay may be compared to those of an otherwise identical sample that has not been contacted with the candidate agent. Such a method may be employed to identify an agent that reduces or increases the abundance of a particular post-translationally modified analyte.

A variety of different candidate agents may be screened by the above methods. Candidate agents encompass numerous chemical classes, though typically they are organic molecules, preferably small organic compounds having a molecular weight of more than 50 and less than about 5000 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

Agents that modulate post-translational modification typically decrease or increase the amount of a post-translationally modified analyte (relative to the total amount of that analyte) by at least about 10%, at least about 20%, at least about 50%, at least about 70%, or at least about 90%.

Kits

Also provided by the subject invention are kits for practicing the subject methods, as described above. The subject kits contain at least a plurality of arrays, e.g., a multi-array substrate having arrays or a plurality of optically addressable arrays, that contain capture agents that specifically bind to post-translationally modified analytes, as described above. The kit may also contain a multi-well plate adapted to operatively engage with the multi-array substrate, and any other reagent, e.g., binding buffer, that may be employed in the above methods. The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., one or more control samples for use in testing the kit.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of sample analysis, comprising:
depositing sub-fractions of a multi-dimensionally fractionated sample into wells of a multi-well substrate;
contacting each of said deposited sub-fractions with an array to produce a plurality of sub-fraction-contacted arrays; and
interrogating said sub-fraction-contacted arrays to identify a sub-fraction containing a post-translationally modified analyte.

2. The method of claim 1, wherein said array is a spatially addressable or optically addressable array.

3. The method of claim 1, wherein:
said contacting includes operably engaging a multi-array substrate with said multi-well substrate; and
wherein said interrogating includes interrogating said multi-array substrate to identify a sub-fraction containing a post-translationally modified analyte.

4. The method of claim 3, wherein said multi-array substrate is a pillar array or a planar array.

5. The method of claim 3, further comprising assessing mass of analytes in said sub-fraction.

6. The method of claim 3, wherein said multi-array substrate contains a plurality of arrays containing a capture agent that specifically binds to a post-translationally modified analyte.

7. The method of claim 6, wherein said capture agent comprise an antibody.

8. The method of claim 7, wherein said antibody binds glycosylated or phosphorylated polypeptides.

9. The method of claim 7, wherein said capture agent comprises an anti-phosphotyrosine, anti-phosphoserine or anti-phosphothreonine antibody.

10. The method of claim 5, wherein said assessing includes assessing said sub-fraction by mass spectrometry.

11. The method of claim 5, wherein said assessing determines a mass of a post-translationally modified analyte.

12. The method of claim 11, wherein said mass identifies said post-translationally modified analyte.

13. The method of claim 1, wherein said method comprises:
fractionating a sample into a set of fractions using a first liquid phase chromatography device;
fractionating said set of fractions into a set of sub-fractions using a second liquid phase chromatography device;
depositing said set of sub-fractions into the wells of a multi-well plate;
contacting each of said deposited sub-fractions with an array to produce a plurality of sub-fraction-contacted arrays; and
interrogating said sub-fraction-contacted arrays to identify a sub-fraction containing a post-translationally modified analyte.; and
assessing mass of analytes in said identified sub-fraction.

14. The method of claim 13, wherein said first or said second liquid phase chromatography device is an ion exchange chromatography device.

15. The method of claim 13, wherein said first or second device is a reverse phase chromatography device.

16. A system for sample analysis, comprising:
a multi-dimensional sample fractionation system for producing sub-fractions of a sample;
a multi-well substrate for receiving said sub-fractions;
a plurality of arrays;
an array reader; and
a system for assessing analyte mass.

17. The system of claim 16, wherein said arrays are optically addressable arrays

18. The system of claim 16, wherein said arrays are on a multi-array substrate.

19. The system of claim 18, wherein said multi-array substrate is a pillar or planar array.

20. The system of claim 15, wherein said system for assessing analyte mass comprises a mass spectrometer system.

21. The system of claim 19, wherein said mass spectrometer system employs a time of flight (TOF) spectrometer, Fourier transform ion cyclotron resonance (FTICR) spectrometer, ion trap, quadrupole or double focusing magnetic electric sector mass analyzer, or any hybrid thereof.

22. A kit comprising:
a plurality of arrays of capture agents that bind to post-translationally modified analytes.

23. The kit of claim 22, wherein said arrays are present on a multi-array substrate.

24. The kit of claim 23, wherein said multi-array substrate is a pillar array.

25. The kit of claim 23, further comprising a multi-well plate adapted to operatively engage with said multi-array substrate.

26. An assay, comprising:
contacting a sample with a candidate agent;
analyzing said sample according to the method of claim 1.

27. The assay of claim 26, wherein said assay is adapted to detect agents that modulate post-translational modification.

28. The assay of claim 26, wherein said assay further comprises analyzing a sample that has not been contacted with said candidate agent.
